(19) United States
(12) Patent Application Publication
(10) Pub. No.: US 2021/0047681 A1
(43) Pub. Date: Feb. 18, 2021

(54) METHODS OF SEQUENCING NUCLEIC ACIDS ON MAGNETIC SENSOR

(71) Applicant: Seagate Technology LLC, Cupertino, CA (US)

(72) Inventors: Gemma MENDONSA, Minneapolis, MN (US); Riyan A. MENDONSA, Minneapolis, MN (US); Krishnan SUBRAMANIAN, Shakopee, MN (US)

(21) Appl. No.: 16/591,162

(22) Filed: Oct. 2, 2019

Related U.S. Application Data
(60) Provisional application No. 62/886,201, filed on Aug. 13, 2019.

(51) Int. Cl. C12Q 1/6869; C12Q 1/6876

(52) U.S. Cl. CPC C12Q 1/6869; B82Y 5/00; C12Q 1/6876

(57) ABSTRACT

Methods of DNA sequencing from a template strand via DNA polymerase immobilized on a magnetic sensor. The methods include forming a complementary strand by the DNA polymerase from individual magnetic nanoparticle (MNP)-labeled nucleotides, and obtaining a signal from the magnetic sensor to identify the MNP and thus the nucleotide. The process can be repeated until the entire complementary strand is formed and identified.

### CROSS-REFERENCE

This application claims priority to U.S. Provisional application No. 62/886,201 filed Aug. 13, 2019, which is incorporated herein by reference for all purposes.

### BACKGROUND

Numerous methods are known for DNA and RNA sequencing. These methods, however, have limitations in sequence read length, sensitivity, and run time. A higher sensitivity or signal/noise ratio would improve sequencing accuracy in long reads. Depending on the method, run times are long due to the need to pause after each base incorporation to obtain an optical signal and/or remove tags.

### SUMMARY

This disclosure is directed to methods of sequencing nucleic acids using polymerase immobilized on a magnetic sensor. Particularly, the methods include bringing nucleotides, labeled with magnetic nanoparticles (MNPs), in close proximity to the magnetic sensor to identity the sequence of nucleotides via an output signal change (e.g., resistance change) sensed by the magnetic sensor. From the output signal, the MNP and thus the nucleotide can be identified.

One particular implementation described herein is a method of sequencing a DNA strand. The method includes providing a template strand, building a complementary strand from a plurality of individual nucleotides A, C, G, T via a DNA polymerase immobilized on a magnetic sensor, each of the individual nucleotides A, C, G, T labeled with a magnetic nanoparticle (MNP) having a different magnetic moment, and identifying, in order, the MNP-labeled nucleotides of the complementary strand by a signal change from the magnetic sensor.

Yet another particular implementation described herein is another method of sequencing a DNA strand. This method includes providing a template strand, providing a plurality of individual nucleotides, each labeled with a corresponding magnetic nanoparticle (MNP), each of the nucleotides identified with a nanoparticle having a different magnetic moment, exposing the template strand and the plurality of individual MNP-labeled-nucleotides to a magnetic sensor having a DNA polymerase immobilized thereon, building a complementary strand from the plurality of MNP-labeled-nucleotides via the polymerase, and identifying, in order, the MNP-labeled nucleotides of the complementary strand by a signal change from the magnetic sensor corresponding to the MNP.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. These and various other features and advantages will be apparent from a reading of the following detailed description.

### BRIEF DESCRIPTION OF THE DRAWING

The described technology is best understood from the following Detailed Description describing various implementations read in connection with the accompanying drawing.

FIG. 1 is a schematic diagram of a DNA strand having nucleotides labeled with magnetic nanoparticles.

FIG. 2 is a schematic diagram of a sequencing sensor and an overall method of the present application.

FIG. 3 is an example graphical representation of signal readout during a sequencing run.

FIG. 4 is a schematic diagram of an array of magnetic sensors having a polymerase immobilized thereon.

FIG. 5 shows an example chemical reaction illustrating a labeled nucleotide incorporated into a growing copy of the template strand via a polymerase.

### DETAILED DESCRIPTION

Current DNA sequencing methods face limitations in sequence read length, sensitivity, and run time. A higher sensitivity or signal/noise ratio would improve sequencing accuracy in long reads. Run times are long due to the need to pause after each base incorporation to obtain an optical signal and/or remove tags, and could be improved with the use of real-time sequencing using a non-optical system. The present invention provides DNA and RNA sequencing methods that overcome these undesired features. It should be noted that although the discussion here may use “DNA” when describing certain features, the methods of this disclosure are also applicable to RNA.

As indicated above, the present disclosure provides methods for real-time sequencing of nucleotides (DNA) utilizing magnetic na.noparticles (MNP) and a magnetic sensor (e.g., a GMR, TMR, AMR, or Hall sensor). The methods include passing nucleotides, labeled with the MNPs, in close proximity to the magnetic sensor and then monitoring a change in a signal from the magnetic sensor (e.g., a resistance change) due to the MNPs affecting the magnetic sensor. MNPs coming into close proximity to some magnetic sensors cause a dramatic change in resistance in the sensor, which can be measured as a signal.

In general, the methods of this disclosure are sequencing a DNA or RNA strand by labeling a plurality of individual nucleotides with one of four different magnetic nanoparticles and identifying each MNP-labeled nucleotide by a signal change from a magnetic sensor. Some of the methods proposed have improved sensitivity compared to optical and electrical detection sequencing methods due to large resistance change caused by MNP-sensor interactions.

Described herein are various methods of and systems for sequencing DNA or RNA via DNA or RNA polymerase immobilized on a magnetic sensor. The method includes, from beginning with a template strand, forming a complementary strand with individual MNP-labeled nucleotides using a DNA polymerase, bringing the MNP-labeled complimentary strand close to the sensor, and obtaining a signal from the sensor identifying the MNP and thus the nucleotide. After identifying the MNP, the MNP is cleaved off of the nucleotide. The process can be repeated until the entire complementary strand is formed and identified.

One particular implementation described herein is a method of sequencing DNA or RNA via polymerase immobilized on a magnetic sensor. The method includes, from beginning with a template strand, forming a complementary strand with MNP-labeled nucleotides, bringing the MNP-labeled complimentary strand close to the sensor, and obtaining a signal from the sensor identifying the MNP and thus the nucleotide. After identifying the MNP, the MNP is cleaved off from the nucleotide. The cleaved MNP diffuses away from the sensor, such that its magnetic moment does not interfere with the signal generated from the subsequently incorporated nucleotide.

Another particular implementation described herein is a method of sequencing a DNA or RNA strand by: providing a template strand; building a complementary strand from a plurality of nucleotides via a polymerase immobilized on a magnetic sensor, each type of nucleotide labeled with a different sized MNP; and identifying, in order, each MNP-labeled nucleotide of the complementary strand by a signal change from the magnetic sensor. After identifying the MNP-labeled nucleotide, the MNP is cleaved off of the nucleotide.

Yet another particular implementation described herein is a method of sequencing a DNA strand by: providing a template strand; providing a plurality of nucleotides, each type of nucleotide labeled with a corresponding MNP, each type of the nucleotides identified with a different sized MNP; exposing the template strand and the plurality of MNP-labeled-nucleotides to a magnetic sensor having a polymerase immobilized thereon; building a stand complementary to the template strand from the plurality of MNP-labeled-nucleotides; and identifying, in order, MNP-labeled nucleotides of the complementary strand by a signal change from the magnetic sensor.

Any of these processes can be repeated for multiple MNP-labeled nucleotides, and even until the entire complementary strand is identified.

The method proposed provides improved sensitivity compared to optical and electrical detection sequencing methods, including fluorescence, due to the large resistance change caused by MNP-sensor interactions. It also offers a short run time, since there are no washing steps, and tag removal happens in real-time.

Another particular implementation described herein is a DNA sequencing device comprising a magnetic sensor having a polymerase immobilized thereon.

In the following description, reference is made to the accompanying drawing that forms a part hereof and in which is shown by way of illustration at least one specific implementation. The following description provides additional specific implementations. It is to be understood that other implementations are contemplated and may be made without departing from the scope or spirit of the present disclosure. The following detailed description, therefore, is not to be taken in a limiting sense. While the present disclosure is not so limited, an appreciation of various aspects of the disclosure will be gained through a discussion of the examples provided below.

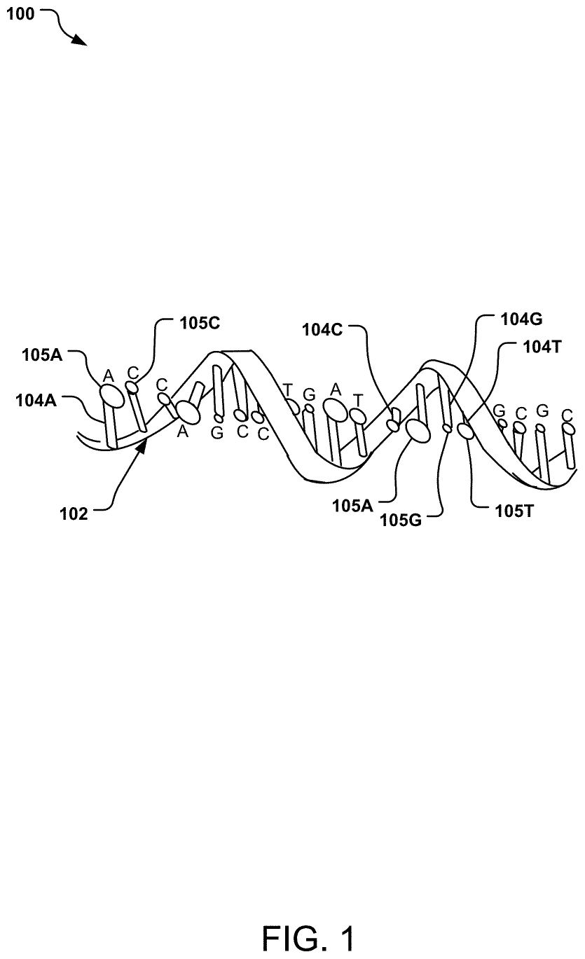

Turning to FIG. 1, for illustration and to provide a general understanding of the elements involved in the methods of this disclosure, a single strand of DNA is illustrated at 100. As all DNA strands, the strand 100 is composed of a backbone 102 and four nucleotides 104, particularly, A, C, G, T, labeled as 104A, 104C, 104G, 104T. As indicated above, the nucleotides 104 are labeled with magnetic nanoparticles (MNPs) depending on their identity. In accordance with the methods described herein, each nucleotide 104A, 104C, 104G, 104T is labeled with its own magnetic nanoparticle (MNP) 105, labeled as 105A, 105C, 105G, 105T. The MNP 105 attaches to the nucleotide 104 at the terminal phosphate of the nucleotide 104.

The MNPs 105 are selected so that each MNP 105A, 105C, 105G, 105T has a different magnet moment. The MNPs may be any magnetic material, e.g., ferromagnetic or paramagnetic. The magnetic moment of the MNPs 105 can differ due to, e.g., the size of the MNPs 105, the shape of the MNPs 105, the composition or material of the MNPs 105, or by insulative coatings that may be present on the MNPs 105. The MNPs 105 exhibit magnetic properties without the presence of an external magnetic field.

When size (e.g., diameter) of the MNPs 105 is the distinguishing feature between the MNPs 105A, 105C, 105G, 105T, typically the size differentiation between the MNPs is at least 10 nm, in some implementations at least 15 nm and in other implementations at least 20 nm. The overall size (e.g., diameter) of the MNPs 105 is, e.g., at least 5 nm and as large as, e.g., 200 nm. In one particular example, each of the four nucleotides 104A, 104C, 104G, 104T, is labeled with a differently-sized magnetic nanoparticle, such as 10 nm, 20 nm, 50 nm, 100 nm (not provided in any order). In another particular example, the four nucleotides 104A, 104C, 104G, 104T are labeled with MNPs of 10 nm, 25 nm, 40 nm, 60 nm (not provided in any order). There is no requirement or recommendation regarding which nucleotide 104A, 104C, 104G, 104T has the largest or the smallest sized MNP.

The MNPs 105A, 105C, 105G, 105T, when they come into close proximity to a magnetic sensor, cause a dramatic change in a signal from or in the sensor, such as a resistance value. In some implementations, the sizes of the MNPs 105A, 105C, 105G, 105T are selected so that a resistance signal from the magnetic sensor, due to interaction with the MNP, falls on a generally-linear portion of the sensor resistance output curve, which is a cosine curve. The magnetic sensor can be any known magnetic sensor (e.g., GMR, TMR, AMR, Hall sensor) and can have any suitable configuration (e.g., CPP, CIP) with any number of ferromagnetic, antiferromagnetic, SAF, pinned, pinning, etc. layers. For GMR, TMR, and AMR sensors, a change in resistance is observed due to the MNP 105. For a Hall sensor, a change in voltage is observed due to the MNP 105.

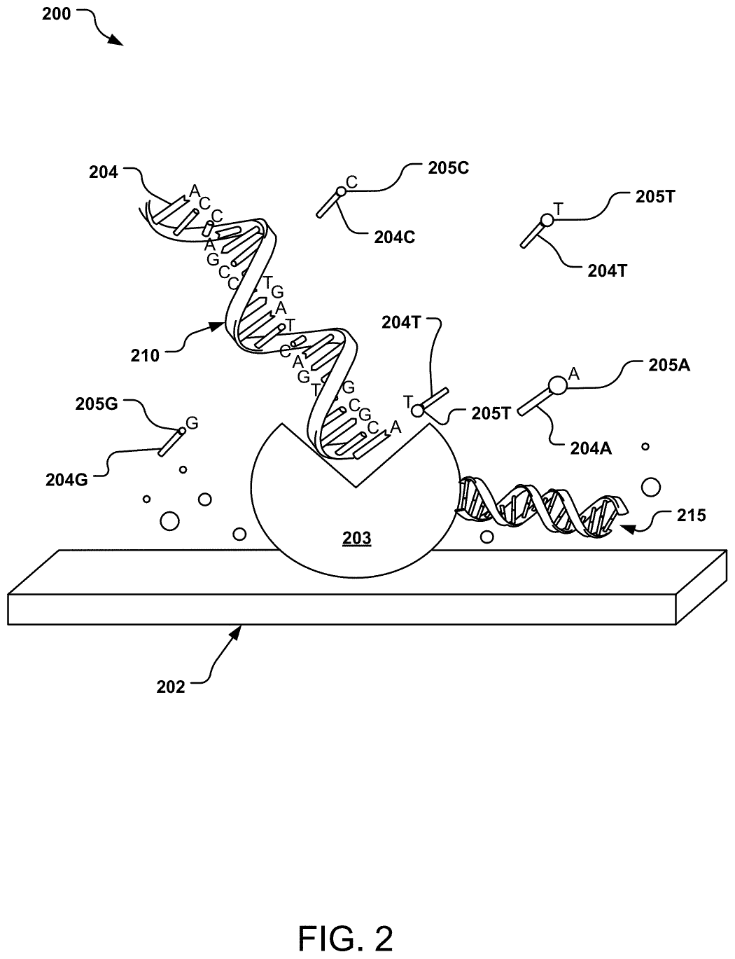

FIG. 2 provides an illustration of a sequencing sensor 200 and its associated method, according to this disclosure, with DNA polymerase immobilized on a magnetic sensor.

The sequencing sensor 200 includes a magnetic sensor (e.g., GMR, TMR, AMR, Hall sensor) 202 having immobilized thereon a DNA polymerase 203 and a plurality of individual nucleotides 204 (labeled with an MNP 205) that are available in a mixture. The polymerase 203 may be, e.g., a continuous even layer of polymerase across the entire sensor or may be present on only portions of the sensor 202.

The DNA polymerase 203 is an enzyme that synthesizes complimentary DNA strands or molecules from nucleotides and a template strand. Any DNA polymerase 203 may be suitable for the application; in some implementations, an RNA polymerase may be used. The DNA polymerase 203 incorporates complementary nucleotides into a growing copy of a template DNA strand 210. The polymerase 203 incorporates one nucleotide 204 at a time, labeled with an MNP 205, building a strand complementary to a template strand 210 (the template strand 210 not labeled with MNPs 205), the nucleotide 204 being complementary to the corresponding nucleotide of the template strand 210.

As seen in FIG. 2 and as previously described in respect to FIG. 1, a plurality of nucleotides 204 are each labeled with an MNP 205, the size (or other magnetic characteristic of the MNP) based on the type of nucleotide 204; each of the four nucleotides 204A, 204C, 204G, 204T is labeled with a differently-sized magnetic nanoparticle MNP 205A, 205C, 205G, 205T. The MNP-labeled-nucleotides are “free” or “unattached,” present in a mixture surrounding the sensor 202 and polymerase 203.

It is noted that, in some implementations, each MNP 205 could have more than one nucleotide 204 attached to it. For example, one MNP could have one A nucleotide attached to it, or it could have, e.g., ten A nucleotides attached to it, or a hundred. In such a situation, each individual nucleotide is still labeled with a different magnetic moment MNP (e.g., different sized MNP), but each individual MNP 205 may have several of the same type of nucleotides attached.

To determine the nucleotide sequence of a DNA or RNA strand, a template strand 210 is provided, of which it is desired to learn the sequence. The template strand 210 can include at least one adapter that may anneal to an appropriate complementary primer.

As the template strand 210 engages with the polymerase 203, an unattached nucleotide 204 complementary to the nucleotide 204 of the template strand 210 at the polymerase 203 forms a portion of the complementary strand; this usually occurs in less than 50 milliseconds (ms), e.g., in less than 25 ms, e.g., in about 14 ms.

Sequentially, unattached nucleotides 204 complementary to the sequence in the template strand 210 attach to the template strand 210 to form the complementary strand. The resulting DNA strand, formed by the template strand 210 and the complementary strand built by the polymerase 203, is shown as strand 215 in FIG. 2.

Essentially immediately after being incorporated into the complementary strand, the MNP 205 is cleaved from its nucleotide 204 by the polymerase 203, releasing the MNP 205 back into the mixture. The free MNPs 205 can be reclaimed and recycled for future use. A magnetic field can be used to facilitate removal of the free MNPs 205 from the mixture.

Each MNP 205, when attached to a nucleotide 204, causes a signal (e.g., resistance) change in the magnetic sensor 202 as it is incorporated by the polymerase 203 into the complementary strand at the magnetic sensor 202. The magnitude of the signal change corresponds to the magnetic property (e.g., size) of the MNP 205 attached to the nucleotide 204. Based on the change in signal from the sensor, the MNP 205, and thus the nucleotide 204 to which it is or was attached, can be identified.

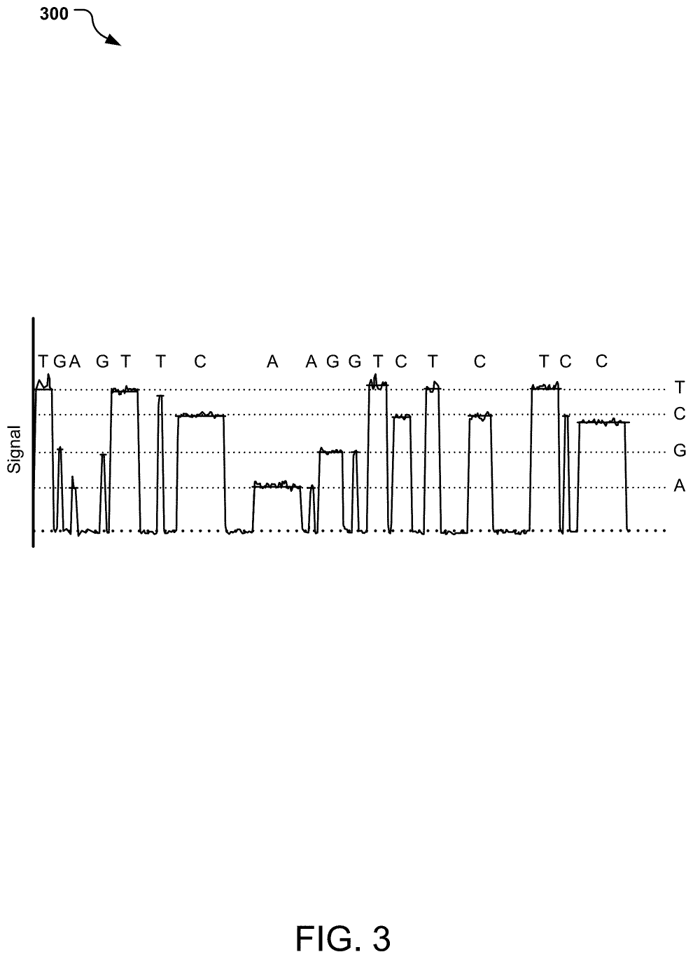

A strand of MNP-labeled nucleotides causes an orderly series of signal changes. An example strand of nucleotides will provide a graph, such as shown in FIG. 3 as graph 300.

Table 1 provides a more detailed description of exemplary steps in the proposed sequencing method.

TABLE 1

| | | |
|---|---|---|
| Step 1 | Library preparation | Ligate adaptor sequences onto the |
| | | ends of template to be sequenced. |
| | | Anneal complimentary primer(s) |
| | | to the adaptor region(s). |
| Step 2 | Immobilize DNA | Sensors may be, e.g., flat, raised, |
| | polymerase onto | or recessed. |
| | magnetic sensor | Multiple sensors (eg., parallel) |
| | | for high throughput sequencing. |
| Step 3 | Add template | DNA polymerase binds to primer + |
| | sequence and | template complex. |
| | MNP-labeled | Nucleotides are labeled to MNPs |
| | nucleotides | via terminal phosphate. Polymerase |
| | | naturally cuts off label during |
| | | incorporation. |
| Step 4 | Sequence the DNA | MNP-labeled nucleotides |
| | | incorporated one at a time. |
| | | Sensor detects MN P signal from |
| | | each incorporation. |
| | | After incorporation, MNP is |
| | | cleaved and diffuses away from |
| | | sensor. |

In Step 1, a template strand is produced with an adapter and an appropriate complementary primer is annealed thereon. In Step 2, a DNA polymerase is immobilized onto a magnetic sensor. In Step 3, the template strand and MNP-labeled nucleotides are combined (e.g., in a liquid mixture) in proximity to the magnetic sensor. It is noted that Steps 1 and 2 may be done in opposite order or simultaneously. In Step 4, the polymerase immobilized on the magnetic sensor incorporates the appropriate nucleotide to build the complementary strand to the template strand. As each MNP-labeled-nucleotide nears or contacts the magnetic sensor, the MNP affects the sensor (e.g., the output of the sensor) and the resulting signal (detected by appropriate measurement equipment) identifies the MNP and thus the nucleotide (e.g., as an A, C, G, T). In such a method, a complementary strand is built with known nucleotides, providing an identification of the initial template strand and thus the DNA strand.

It is noted that, as indicated in Step 4, the MNP is cleaved from the nucleotide by the polymerase after detection of the MNP by the sensor. The cleaved MNPs diffuse and can be moved through, and thus removed from, the mixture by use of a magnetic field. This enables the recycling of MNPs after each sequencing round, saving on materials and cost.

In one specific example method, the DNA polymerase turnover rate is approximately 14 ms per nucleotide, although rates of about 10 ms-50 ms may occur. MNP detection will occur during this time before the polymerase releases the incorporated nucleotide and the cleaved MNP.

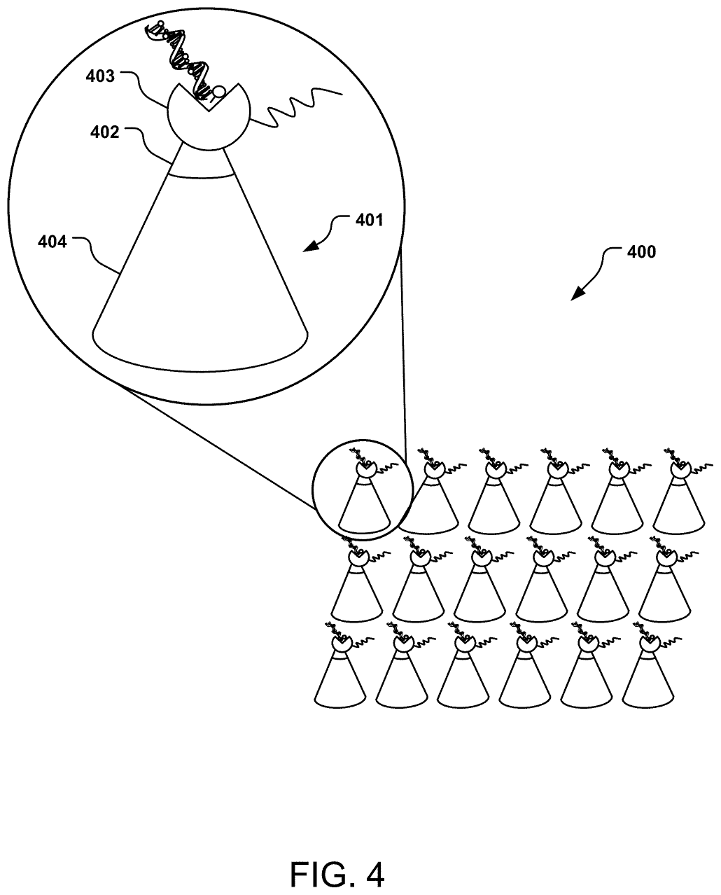

To facilitate the diffusion and removal of the released MNPs away from the magnetic sensor, the sensor may have an elevated height (e.g., as compared to a flat sensor as shown in FIG. 2). FIG. 4 is a schematic diagram of a sequencing system 400 that has an array of individual sequence sensors 401 shown on raised supports.

Each sequencing sensor 401 has a magnetic sensor 402 with a DNA polymerase 403 immobilized thereon seated or otherwise attached to a support structure 404. The raised support structure 404 facilitates removal of the MNPs from the magnetic sensor surface, e.g., due to gravity.

The support structure 404 may be any suitable pedestal-like structure, e.g., columnar, conical, pyramidal, etc. The top of the support structure 404 may be flat (e.g., truncated) or may be a pointed tip. Alternately, the sensor 402 may be situated, e.g., recessed, in a microwell or other retaining structure on the support structure 404. The polymerase 403 may cover all or part of the top of the support structure 404.

Multiple sequencing sensors, as in the system 400 of FIG. 4, may be utilized simultaneously to provide parallel sequencing. Any multiple sequencing sensors, whether on a flat support (as in FIG. 2), raised (as in FIG. 4), recessed or otherwise situated, may be orderly arranged (e.g., in columns and/or rows) or may be randomly arranged; multiple sequencing sensors may be arranged in a plane (planar) or not.

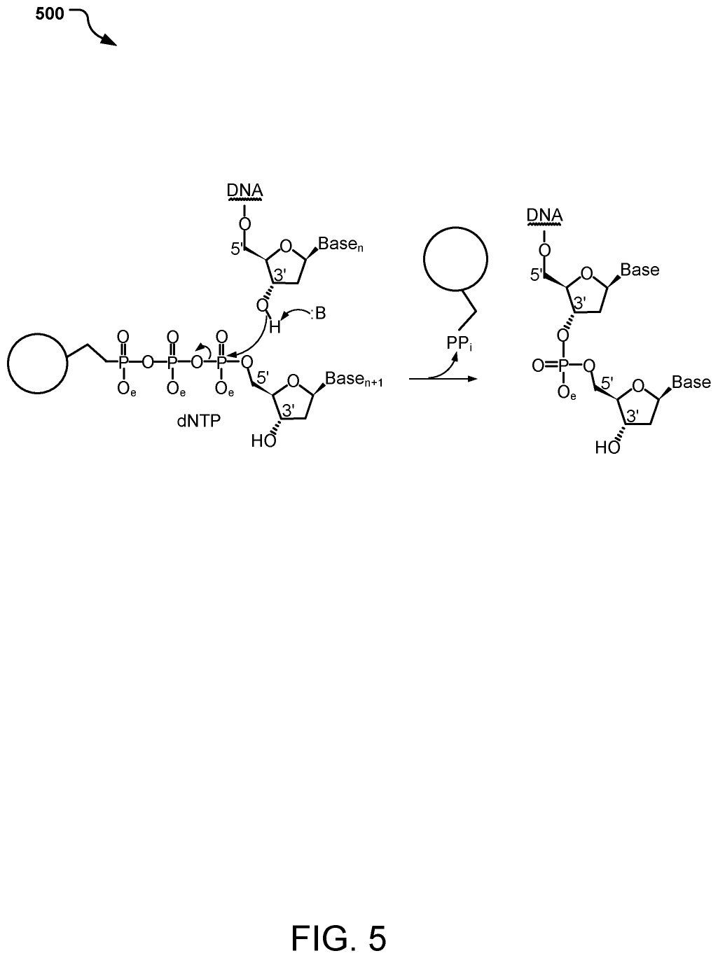

FIG. 5 illustrates an example reaction for an MNP-labeled nucleotide. FIG. 5 shows an example chemical reaction that may take place as the polymerase incorporates the labelled nucleotide into a growing copy of the template strand. If the label is located on the terminal phosphate, for example, the polymerase will cleave it away during incorporation, releasing the tag.

In this example, the MNP is connected to the nucleotide via the terminal phosphate of the nucleotide. The linker between the nucleotide and the MNP may be any polymeric entity, such as polyethylene glycol. During nucleotide incorporation via the DNA polymerase, the terminal phosphates are cleaved off by the polymerase, releasing the MNP and at least a portion of the linker.

The above specification and examples provide a complete description of the structure and use of exemplary implementations of the invention for sequencing. The above description provides specific implementations. It is to be understood that other implementations are contemplated and may be made without departing from the scope or spirit of the present disclosure. The above detailed description, therefore, is not to be taken in a limiting sense. While the present disclosure is not so limited, an appreciation of various aspects of the disclosure will be gained through a discussion of the examples provided.

Unless otherwise indicated, all numbers expressing feature sizes, amounts, and physical properties are to be understood as being modified by the term “about,” whether or not the term “about” is immediately present. Accordingly, unless indicated to the contrary, the numerical parameters set forth are approximations that can vary depending upon the desired properties sought to be obtained by those skilled in the art utilizing the teachings disclosed herein.

As used herein, the singular forms “a”, “an”, and “the” encompass implementations having plural referents, unless the content clearly dictates otherwise. As used in this specification and the appended claims, the term “or” is generally employed in its sense including “and/or” unless the content clearly dictates otherwise,

Spatially related terms, including but not limited to, “bottom,” “lower”, “top”, “upper”, “beneath”, “below”, “above”, “on top”, “on,” etc., if used herein, are utilized for ease of description to describe spatial relationships of an element(s) to another. Such spatially related terms encompass different orientations of the device in addition to the particular orientations depicted in the figures and described herein. For example, if a structure depicted in the figures is turned over or flipped over, portions previously described as below or beneath other elements would then be above or over those other elements.

Since many implementations of the invention can be made without departing from the spirit and scope of the invention, the invention resides in the claims hereinafter appended. Furthermore, structural features of the different implementations may be combined in yet another implementation without departing from the recited claims.

What is claimed is:

1. A method of sequencing a DNA strand, comprising:
providing a template strand;
building a complementary strand from a plurality of individual nucleotides A, C, G, T via a DNA polymerase immobilized on a magnetic sensor, each of the individual nucleotides A, C, G, T labeled with a magnetic na.noparticle (MNP) having a different magnetic moment; and
identifying, in order, the MNP-labeled nucleotides of the complementary strand by a signal change from the magnetic sensor.

2. The method of claim 1, further comprising:
after identifying the MNP-labeled nucleotide, cleaving the MNP from the nucleotide.

3. The method of claim I, wherein the magnetic sensor is a GMR sensor, a TMR sensor, or an AMR sensor.

4. The method of claim 3, where the signal change is a resistance change.

5. The method of claim 1, comprising a plurality of magnetic sensors having DNA polymerase immobilized thereon.

6. The method of claim I, wherein each individual nucleotide is labeled with a different-size magnetic nanoparticle.

7. A method of sequencing a DNA strand, comprising:
providing a template strand;
providing a plurality of individual nucleotides, each labeled with a corresponding magnetic nanoparticle (MNP), each of the nucleotides identified with a nanoparticle having a different magnetic moment;
exposing the template strand and the plurality of individual MNP-labeled-nucleotides to a magnetic sensor having a DNA polymerase immobilized thereon;
building a complementary strand from the plurality of MNP-labeled-nucleotides via the polymerase; and
identifying, in order, the MNP-labeled nucleotides of the complementary strand by a signal change from the magnetic sensor corresponding to the MNP.

8. The method of claim 7, further comprising:
after identifying the MNP-labeled nucleotide, cleaving the MNP from the nucleotide.

9. The method of claim 7, wherein the magnetic sensor is a GMR sensor, a TMR sensor, or an AMR sensor.

10. The method of claim 9, where the signal change is a resistance change.

11. The method of claim 7, comprising a plurality of magnetic sensors having DNA polymerase immobilized thereon.

12. The method of claim 7, wherein each individual nucleotide is labeledwith a different-size magnetic nanoparticle.

13. A DNA or RNA sequencing system comprising a magnetic sensor having a DNA polymerase immobilized thereon.

14. The sequencing system of claim 13 comprising an array of a plurality of magnetic sensors having DNA polymerase immobilized thereon.

15. The sequencing system of claim 13, wherein the magnetic sensor is on a raised support structure.
